(19) United States
(12) Patent Application Publication
(10) Pub. No.: US 2023/0280331 A1
(43) Pub. Date: Sep. 7, 2023

(54) Device And Method For Determining The Haemoglobin Or Haematocrit Level Of A Flowing Liquid

(71) Applicant: I-SEP, Nantes (FR)

(72) Inventors: Stéphane Chollet, La Chapelle Sur Erdre (FR); Gaëtan Coulon, Baden (FR); Benoît Decouture, Nantes (FR); Sylvain Picot, Caluire Et Cuire (FR); Patricia Forest-Villegas, Genas (FR); Francis Gadrat, Bordeaux (FR)

(73) Assignee: I-SEP, Nantes (FR)

(21) Appl. No.: 18/004,399

(22) PCT Filed: Jul. 8, 2021
(86) PCT No.: PCT/FR2021/051272

§ 371 Date: Jan. 5, 2023

(30) Foreign Application Priority Data
Jul. 8, 2020 (FR) FR2007200
Jul. 8, 2020 (FR) FR2007201

(51) Int. Cl. G01N 33/49; G01N 21/84; G01N 21/25; A61M 1/36

(52) U.S. Cl. CPC G01N 33/4915; G01N 21/84; G01N 21/255; A61M 1/3609; A61M 2205/3306; A61M 2230/207; G01N 2201/069

(57) ABSTRACT

The invention relates to a device and method for determining the haematocrit and/or haemoglobin level of a liquid flowing in a tubular portion (2), the method comprising:—emitting light beams in the direction of the tubular portion (2) with at least two light sources (11; 21), each of the two light sources (11; 21) being configured to emit light beams at an emission wavelength chosen to correspond to an isobestic point of the haemoglobin;—receiving light signals transmitted through the tubular portion (2) with at least two light sensors (12; 22), each light sensor (12; 22) being associated with one of the two light sources (11; 21);—calculating the haematocrit or haemoglobin level in the liquid by processing the light signals received by the light sensors (12; 22); characterised in that the emission power of at least one of the light sources (11; 21) is modified while the haematocrit and/or the haemoglobin level is determined according to the haematocrit and/or respectively the haemoglobin level calculated for the liquid.

### TECHNICAL FIELD

The present disclosure relates to an apparatus and a method for the determination of a blood parameter of a circulating fluid, particularly for the determination of the hemoglobin level and/or the hematocrit level of the circulating fluid. The present disclosure finds a particularly advantageous application for medical applications, for example for the analysis of hemorrhagic fluid circulating in a tube.

### STATE OF THE ART

There are many medical applications for which the tracking of the blood parameters for qualifying the hemorrhagic fluid of the patient is necessary. Tracking the evolution of the hemoglobin level and/or the hematocrit level of a hemorrhagic fluid can for example be useful in some procedures, in particular surgical procedures. Particularly, when the hemorrhagic fluid of a patient must undergo a specific treatment, it is advantageous to be able to continuously track the evolution of these blood parameters.

One particular and non-limiting example where it is useful to be able to track the evolution of blood parameters is the treatment of hemorrhagic fluid for a blood autotransfusion in a patient. The autotransfusion or autologous transfusion, i.e. the transfusion in a patient with their own blood, is increasingly practiced during surgical procedures, since it avoids the incompatibilities that may occur during homologous or allogeneic transfusions. The autotransfusion moreover avoids the transmission of infectious diseases.

For a proper operation of these hemorrhagic fluid treatment systems, it is imperative to be able to track in real time the evolution of the hemoglobin concentration or the hematocrit level in the fluid being treated since the evolution of the blood parameters of this fluid can allow driving the treatment system. One of the difficulties lies in the fact that the fluid whose hemoglobin and/or hematocrit level is to be known is circulating, generally in flexible tubing, which complicates the detection. Furthermore, it is necessary to apply specific detection methods to compensate for the losses in the detection sensitivity due to the movement of the fluid. Moreover, since the hemorrhagic fluid treatment systems (in particular for autotransfusion) are generally used in emergency situations, it is important that the entire system can be used immediately, by avoiding as much as possible any preliminary calibration step, including with regard to the component that allows determining the hemoglobin and/or hematocrit level of the hemorrhagic fluid to be treated.

In the article entitled “Noninvasive and Continuous Hematocrit Measurement by Optical Method without Calibration” published by SHOTA EKUNI and YOSHIYUKI SANKAI in “Electronics and Communications in Japan, Vol. 99, No. 9, 2016”, an optical detection method and system have been proposed for the determination of the hematocrit level of a fluid circulating in a tube and avoiding a prior calibration of the detection system. The proposed optical system consists of two transceiver assemblies operating in backscatter, each transceiver assembly being arranged on a respective support. The two supports are provided to be attached to each other while surrounding a tubular portion through which the fluid whose hematocrit level is to be determined circulates, without however deforming this tubular portion. The two transceiver assemblies operate according to different wavelengths corresponding to isosbestic points of hemoglobin, namely at 810 nm and 1,300 nm, and alternately to avoid interference in the measurements. According to this article, the distance between the transmitter and the receiver has a significant influence on the reliability of the determination of the hematocrit level and it therefore appears necessary to keep it as low as possible (less than 4 mm). In practice, this leads to significant constraints in terms of manufacture and application.

### DISCLOSURE OF THE INVENTION

One aim of the present invention is to propose an apparatus for determining a blood parameter such as the hemoglobin concentration (also called hemoglobin level) and/or the hematocrit level which can be used for a fluid circulating in a tubular portion having any diameter, in particular a flexible tube used in a medical environment.

Another aim of the invention is to propose an apparatus for determining the hemoglobin level and/or the hematocrit level of a fluid circulating in a tubular portion having increased reliability, and in particular allowing measurements in a wide range of the levels. For example, one aim is to allow hematocrit level measurements both for low hematocrit levels that is to say lower than or equal to 30%, and for high hematocrit levels that is to say higher than 30%. Particularly, one aim of the present invention is to propose an apparatus for determining hematocrit levels at least within the range from 5% to 60%, and in particular between 20% and 50%.

Another aim of the present invention is to propose an apparatus for determining the hemoglobin level and/or the hematocrit level of a fluid circulating in a tubular portion which can be positioned on this tubular portion in a simple manner, without having in particular to modify this tubular portion, and without having to stop the circulation of the fluid if necessary. Advantageously, the apparatus for determining the hemoglobin level and/or the hematocrit level proposed can be directly used on a fluid treatment system, for example a hemorrhagic fluid treatment system for autotransfusion, by using the pre-existing tubing in the treatment system, without having to dismount the elements of this treatment system in particular.

Another aim of the present invention is to propose an apparatus for determining the hemoglobin level and/or the hematocrit level which can be used for a fluid circulating in a tubular portion at a high flow rate (typically greater than 1,000 ml/min, for example of the order of 2,000 ml/min) without however substantially disturbing the flow rate of this circulating fluid, to avoid possible harmful effects on the fluid, for example to avoid creating hemolysis for a circulation of hemorrhagic fluid.

Another aim of the present invention is to propose a method for determining the hemoglobin level and/or the hematocrit level of a fluid circulating in a tubular portion that is reliable and simple to implement, and allowing measurements in a wide range of levels, particularly both for low hematocrit levels that is to say lower than or equal to 30%, and for high hematocrit levels that is to say higher than 30%. Particularly, one aim of the present invention is to propose a method for determining hematocrit levels at least within the range from 5% to 60%, and in particular between 20% and 50%.

To this end, an apparatus for determining the hematocrit level and/or the hemoglobin level of a fluid circulating in a tubular portion is proposed, comprising:
two transceiver assemblies, each transceiver assembly comprising a light source and a light sensor provided to be arranged on either side of the tubular portion at a fluid circulation area for a measurement in transmission, preferably through curved walls of the tubular portion;
the light source of each of the two transceiver assemblies being configured to emit light beams according to an emission wavelength chosen to correspond to an isosbestic point of hemoglobin;
each transceiver assembly further comprising a collimation system for collimating the light beam emitted from the corresponding light source in the direction of the corresponding light sensor.

An apparatus for determining the hematocrit level and/or the hemoglobin level of a fluid circulating in a tubular portion is also proposed, comprising:
two transceiver assemblies, each transceiver assembly comprising a light source and a light sensor provided to be arranged on either side of the tubular portion at a fluid circulation area for a measurement in transmission, preferably through curved walls of the tubular portion;
the light source of each of the two transceiver assemblies being configured to emit light beams according to an emission wavelength chosen to correspond to an isosbestic point of hemoglobin;
a processing system programmed to determine the hematocrit level and/or the hemoglobin level of the fluid as a function of the light signals received by the light sensors of the transceiver assemblies; and
a monitoring system comprising means for modifying the power emitted by the light sources, the monitoring system being programmed to modify the emission power of the light sources as a function of the hematocrit level and/or the hemoglobin level determined for the fluid.

Preferred but non-limiting aspects of either of these apparatuses, taken alone or in combination, are as follows:
the apparatus comprises a support assembly on which the two transceiver assemblies are mounted, the support assembly being configured to be positioned around the tubular portion;
the respective light sources of the two transceiver assemblies are configured to emit light beams at two different emission wavelengths;
at least one of the light sources of the transceiver assemblies is configured to emit light beams according to an emission wavelength chosen for an absorption of the light beams substantially identical in water or in plasma;
at least one, and preferably each, collimation system comprises an upstream lens(es) assembly having a focal plane and being positioned between the corresponding light source and light sensor on the side of the light source with respect to the tubular portion, the light source being positioned at more or less 10 mm from the focal plane of the upstream lens(es) assembly, and preferably in the focal plane of the upstream lens(es) assembly;
at least one, and preferably each, collimation system comprises a downstream lens(es) assembly having a focal plane and being positioned between the corresponding light source and light sensor on the side of the light sensor with respect to the tubular portion, the light sensor being positioned at more or less 10 mm from the focal plane of the downstream lens(es) assembly, and preferably in the focal plane of the downstream lens(es) assembly;
at least one, and preferably each, collimation system comprises a downstream lens(es) assembly having a focal plane and being positioned between the corresponding light source and light sensor on the side of the light sensor with respect to the tubular portion, the downstream lens(es) assembly is positioned so that the light beams leaving the outlet wall of the tubular portion converge at more or less 10 mm from the focal plane of the downstream lens(es) assembly, and preferably in the focal plane of the downstream lens(es) assembly;
at least one, and preferably each, collimation system comprises an upstream diaphragm positioned between the corresponding light source and light sensor on the side of the light source with respect to the tubular portion, the upstream diaphragm being provided to let pass a central portion of the light beams emitted by the light source in the direction of the light sensor and to stop a peripheral portion of the light beams emitted by the light source;
at least one, and preferably each, collimation system comprises a downstream diaphragm positioned between the corresponding light source and light sensor on the side of the light sensor with respect to the tubular portion, the downstream diaphragm being provided to let pass a central portion of the light beams transmitted through the tubular portion in the direction of the light sensor and to stop a peripheral portion of the light beams transmitted through the tubular portion;
at least one, and preferably each, collimation system comprises an upstream filter positioned between the corresponding light source and light sensor on the side of the light source with respect to the tubular portion, and/or a downstream filter positioned between the corresponding light source and light sensor on the side of the light sensor with respect to the tubular portion, the upstream and downstream filters of the collimation system of a transceiver assembly being provided to filter at least the emission wavelength of the light source of the other transceiver assembly;
the apparatus is such that the light source of a first of the two transceiver assemblies is configured to emit light beams at a wavelength comprised between 780 nm and 840 nm, preferably comprised between 800 nm and 820 nm, and more preferably equal to 810 nm; and the light source of a second of the two transceiver assemblies is configured to emit light beams at a wavelength comprised between 1,270 nm and 1,330 nm, preferably comprised between 1,290 nm and 1,310 nm, and more preferably equal to 1,300 nm;
the light sources of the transceiver assemblies are positioned on the same side with respect to the tubular portion;
the apparatus further comprises a system for monitoring the transceiver assemblies, the monitoring system comprising means for synchronizing the light sources and/or means for modifying the power emitted by the light sources;
the transceiver assemblies are assembled on a single support having a groove intended to receive the tubular portion;
the apparatus further comprises a cover provided to at least partially cover the groove, said cover comprising a compression portion intended to hold in position the tubular portion positioned in the groove;
the light sources and all elements of the transceiver assemblies provided to be on the side of the corresponding light source with respect to the tubular portion are assembled on an upstream support, and the light sensors and all elements of the transceiver assemblies provided to be on the side of the corresponding light sensor with respect to the tubular portion are assembled on a downstream support distinct from the upstream support, the downstream and upstream supports having complementary shapes provided to be coupled so as to enclose the tubular portion;
the apparatus comprises a system for deforming the tubular portion facing the transceiver assemblies, the deformation system being provided to deform a circular section of the tubular portion into an ellipsoidal section;
the light sources and all elements of the transceiver assemblies provided to be on the side of the corresponding light source with respect to the tubular portion are positioned on one side of a major axis defining the ellipsoidal section, and the light sensors and all elements of the transceiver assemblies provided to be on the side of the corresponding light sensor with respect to the tubular portion are positioned on the other side of the major axis defining the ellipsoidal section;
the ellipsoidal section is defined by a major radius (Ra) along the major axis and by a minor radius (Rb) along a minor axis perpendicular to the major axis, the ellipsoidal section having, in a deformed state of the portion tubular, a small radius (Rb) having a length comprised between 30% and 70%, and preferably of the order of 50%, of the radius of the circular section of the tubular portion in the undeformed state.

A method for determining the hematocrit level and/or the hemoglobin level of a fluid circulating in a tubular portion is further proposed, comprising:
the emission of light beams in the direction of the tubular portion with at least two light sources, each of the two light sources being configured to emit light beams according to an emission wavelength chosen to correspond to an isosbestic point of hemoglobin;
the receipt of light signals transmitted through the tubular portion with at least two light sensors, each light sensor being associated with one of the two light sources;
the calculation of the hematocrit level and/or the hemoglobin level of the fluid by a processing of the light signals received by the light sensors;
characterized in that the emission power of at least one of the light sources is modified during the determination of the hematocrit level and/or the hemoglobin level as a function of the hematocrit level and/or respectively of the hemoglobin level calculated for the fluid.

Preferred but non-limiting aspects of this method, taken alone or in combination, are as follows:
the emission power used for the light sources is at most equal to 100% of the maximum emission power of said light sources, and preferably comprised between 10% and 60% of the maximum emission power of said light sources;
the emission power of the light sources is monitored independently for each of the light sources;
the emission power of at least one of the light sources is increased from a threshold value of the hematocrit level and/or the hemoglobin level calculated for the fluid;
the method is such that:
   the emission power of the light source is set to a value comprised between 10% and 30%, preferably equal to 20%, of the maximum emission power of said light source when the calculated hematocrit level is lower than 30%; and
   the emission power of the light source is set to a value comprised between 30% and 100%, preferably equal to 55%, of the maximum emission power of said light source when the calculated hematocrit level is higher than or equal to 30%;
the emission power of at least one of the light sources is adjusted so that:
   the emission power of said light source is at a first power level for values of the hematocrit level calculated for the fluid lower than a first threshold value;
   the emission power of said light source is at a second power level for values of the hematocrit level calculated for the fluid higher than or equal to the first threshold value but lower than a second threshold value higher than the first threshold value; and
   the emission power of said light source is at a third power level for values of the hematocrit level calculated for the fluid higher than or equal to the second threshold value;
the emission power of at least one of the light sources is adjusted so that:
   the emission power of the light source is set to a value comprised between 5% and 15%, preferably equal to 10%, of the maximum emission power of said light source when the calculated hematocrit level is lower than 20%;
   the emission power of the light source is set to a value comprised between 15% and 30%, preferably equal to 20%, of the maximum emission power of said light source when the calculated hematocrit level is comprised between 20% and 30%; and
   the emission power of the light source is set to a value comprised between 30% and 100%, preferably equal to 55%, of the maximum emission power of said light source when the calculated hematocrit level is higher than or equal to 30%.
the emission power of the light sources is modified during the determination of the hematocrit level and/or the hemoglobin level depending on the presence or absence of fluid in the tubular portion and/or on the nature of said fluid;
the light sources are monitored to emit light beams concomitantly.

### DESCRIPTION OF THE FIGURES

Other characteristics and advantages of the invention will emerge from the following description, which is purely illustrative and not limiting and should be read in relation to the appended drawings, in which:

FIG. 1 schematically illustrates the arrangement and operation of the apparatus proposed for the determination of the hematocrit level and/or the hemoglobin level of a circulating fluid, according to a first exemplary embodiment.

FIG. 2 schematically illustrates the arrangement and operation of the apparatus proposed for the determination of the hematocrit level and/or the hemoglobin level of a circulating fluid, according to a second exemplary embodiment.

FIG. 3 schematically illustrates the arrangement and operation of the apparatus proposed for the determination of the hematocrit level and/or the hemoglobin level of a circulating fluid, according to a third exemplary embodiment.

FIG. 4 schematically illustrates the arrangement and operation of the apparatus proposed for the determination of the hematocrit level and/or the hemoglobin level of a circulating fluid, according to a fourth exemplary embodiment.

FIG. 5 illustrates the transceiver assemblies (10; 20) of the proposed apparatus, mounted on a support.

FIG. 6 is a perspective view of the apparatus proposed for the determination of the hematocrit level and/or the hemoglobin level of a circulating fluid.

FIG. 7 illustrates the mounting of the collimation systems in the mounting shafts provided for the assembly of the transceiver assemblies.

FIG. 8 illustrates the mounting of the mounting shafts provided for the assembly of the transceiver assemblies on the support.

### DETAILED DESCRIPTION OF THE INVENTION

The remainder of the description will deal with the determination of the hematocrit level of a circulating fluid but the teachings can be applied for other types of blood parameters such as the hemoglobin level for example.

Apparatus for the Determination of the Hematocrit Level of a Circulating Fluid

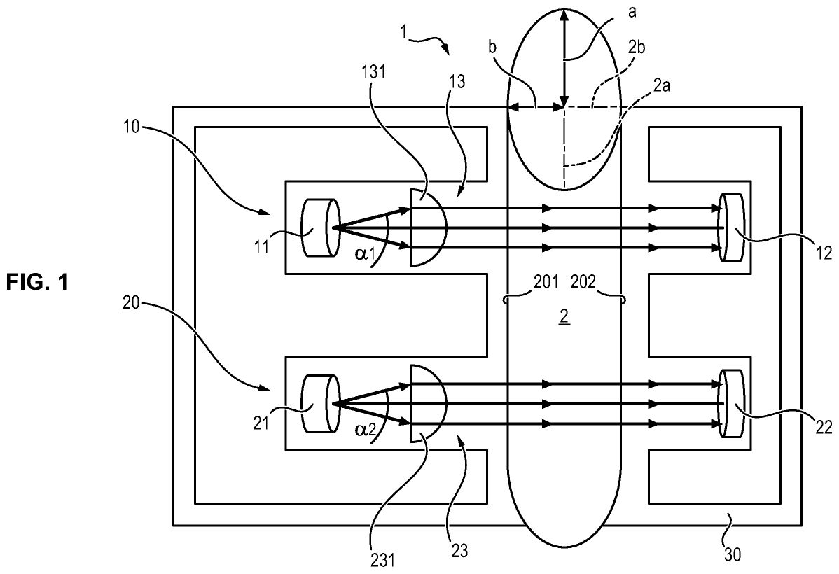

FIG. 1 schematically represents the arrangement of the apparatus 1 proposed for the determination of the hematocrit level of a fluid circulating in a tubular portion 2.

The apparatus 1 for determining the hematocrit level proposed can be used to determine the hematocrit level of any type of fluid but it is particularly adapted to determine the hematocrit level of a hemorrhagic fluid such as human blood, circulating in a tubing, for example a flexible tube used in a standard manner in a hospital environment.

As will be detailed below, the proposed apparatus 1 allows determining the hematocrit level of a fluid in a non-invasive manner that is to say without having to intervene on the fluid as such, which can therefore continue to circulate freely in the tubing.

The proposed apparatus 1 comprises at least two transceiver assemblies (10; 20), each transceiver assembly (10; 20) comprising a light source (11; 21) and a light sensor (12; 22). These two transceiver assemblies (10; 20) are used to determine the hematocrit level of the fluid circulating in the tubing, the light beams passing through the fluid to be analyzed being used to calculate the hematocrit level of the fluid. The fact of having two transceiver assemblies (10; 20) allows increasing the reliability of the apparatus 1 since the measurements of the two light sensors (12; 22) can be correlated. Furthermore, this allows having redundancy which can be advantageous in case of failure of one of the two transceiver assemblies (10; 20).

The light source (11; 21) of each of the two transceiver assemblies (10; 20) is configured to emit light beams according to an emission wavelength chosen to correspond to an isosbestic point of hemoglobin. It is understood that an isosbestic point corresponds to a wavelength value at which the total absorbance of a sample remains constant during a chemical reaction or a possible change of state of this sample. More specifically, an isosbestic point is a wavelength (λᵢₛₒ) at which the total absorbance of a chromophore remains constant regardless of the state it is in. At this precise point, several chromophores have the same molar extinction coefficient (α(λᵢₛₒ)).

There are several isosbestic points for hemoglobin.

For example, the oxyhemoglobin and the deoxyhemoglobin have isosbestic points at 550 nm, 570 nm and near 810 nm of wavelength. At these wavelengths (λᵢₛₒ), a measurement linked to the total volume of hemoglobin can therefore be obtained, since the absorption of light at this wavelength is independent of the oxygenated or reduced state in which the hemoglobin is in.

Furthermore, a wavelength near 1,300 nm corresponds to another isosbestic point of hemoglobin.

The wavelengths above 1,400 nm are also generally isosbestic wavelengths of hemoglobin, particularly the wavelengths comprised between 1,400 nm and 2,200 nm. One advantage of these specific wavelengths is that the absorption of light at these wavelengths is substantially the same in water and in plasma. Thus, the hematocrit level determined at these wavelengths will be the same regardless of the matrix carrying the red blood cells, whether this matrix is mainly composed of plasma or whether it is mainly composed of water. This is particularly true for the wavelengths comprised between 1,400 nm and 1,700 nm and comprised between 1,900 nm and 2,200 nm. Such wavelengths are therefore particularly advantageous when the hemorrhagic fluid for which the hematocrit level is to be determined is diluted more or less strongly with an aqueous solution, such as a saline solution for example.

Thus, one of the light sources (11; 21) of the transceiver assemblies (10; 20) can be for example configured to emit light beams at a wavelength comprised between 780 nm and 840 nm, preferably a wavelength comprised between 800 nm and 820 nm, and more preferably a wavelength equal to 810 nm.

One of the light sources (11; 21) of the transceiver assemblies (10; 20) can be for example configured to emit light beams at a wavelength comprised between 1,270 nm and 1,330 nm, preferably a wavelength comprised between 1,290 nm and 1,310 nm, and more preferably a wavelength equal to 1,300 nm.

One of the light sources (11; 21) of the transceiver assemblies (10; 20) can be for example configured to emit light beams at a wavelength comprised between 1,450 nm and 1,550 nm, preferably a wavelength comprised between 1,490 nm and 1,510 nm, and more preferably a wavelength equal to 1,500 nm.

One of the light sources (11; 21) of the transceiver assemblies (10; 20) can be for example configured to emit light beams at a wavelength comprised between 530 nm and 620 nm, preferably a wavelength comprised between 550 nm and 600 nm, and more preferably a wavelength equal to 550 nm, 570 nm or 590 nm.

The respective light sources (11; 21) of the two transceiver assemblies (10; 20) can be configured to emit light beams at two identical emission wavelengths, but it is advantageous that the two light sources (11 21) are configured to emit light beams at two different emission wavelengths. In addition to the advantage mentioned above due to the functional redundancy of the two transceiver assemblies (10; 20), the use of two light sources operating at different wavelengths allows better correlation of the measurements with a view to calculating the hematocrit level of the fluid circulating in the tubular portion 2.

According to one particular example, one of the light sources (11; 21) of the transceiver assemblies (10; 20) is configured to emit light beams at a wavelength comprised between 780 nm and 840 nm, preferably a wavelength comprised between 800 nm and 820 nm, and more preferably a wavelength equal to 810 nm, while the other light source (11; 21) is configured to emit light beams at a wavelength comprised between 1,270 nm and 1,330 nm, preferably a wavelength comprised between 1,290 nm and 1,310 nm, and more preferably a wavelength equal to 1,300 nm. Light-emitting diodes (LED) can be for example used such as the one proposed by the company “THORLABS” under the reference LED810L (for a light source at 810 nm) and the one proposed by the company “MARKTECH Optoelectronics” under the reference MTE1300NN1-WRC (for a light source at 1,300 nm).

As illustrated in FIG. 1, the light source (11; 21) and the light sensor (12; 22) of each transceiver assembly (10; 20) are provided to be arranged on either side of the tubular portion 2 at a fluid circulation area, which forms a detection area for the transceiver assemblies (10; 20). Such an arrangement will allow a measurement in transmission, that is to say the light beams coming from each of the light sources (11; 21) are intended to pass through the fluid in circulation in the tubular portion 2 before reaching the light sensors (12; 22) of the corresponding transceiver assembly (10; 20). More specifically, each light beam coming from the light sources (11; 21) passes through a first wall of the tubular portion 2, called inlet wall 201, then passes through the fluid circulating in the tubular portion 2, then passes through a second wall of the tubular portion 2, opposite to the inlet wall 201 and called outlet wall 202.

According to one advantageous embodiment, the light sources (11; 21) of the transceiver assemblies (10; 20) are positioned on the same side with respect to the tubular portion 2. This allows in particular facilitating the mounting of the different elements forming the apparatus 1 for determining the hematocrit level and improves the compactness of the apparatus 1 since the similar elements and therefore of the same size are placed on the same side.

Advantageously, each transceiver assembly (10; 20) further comprises a collimation system (13; 23) provided for a collimation of the light beam emitted from the corresponding light source (11; 21) in the direction of the associated light sensor (12; 22).

More specifically, such collimation systems (13; 23) are configured for an infinite collimation of the light beams coming from the light sources (11; 21) in the direction of the tubular portion 2.

When the light beams coming from the light sources (11; 21) pass through the tubular portion 2 in which the fluid circulates, the optical path of these light beams is modified by the crossing of the inlet wall 201 of the tubular portion 2, by the crossing of the fluid circulating in the tubular portion 2, then by the crossing of the inlet wall 202 of the tubular portion 2. The fact of collimating to infinity the light beams coming from the light sources (11; 21) allows converging the light beams in the direction of the light sensors (12; 22) of the transceiver assemblies (10; 20) whatever the shape of the tubular portion, particularly if this tubular portion 2 is not deformed with a circular section or if this tubular portion 2 is deformed with an ellipsoidal section.

Each collimation system (13; 23), or at least one of the two, can for example comprise an upstream lens(es) assembly (131; 231) having a focal plane and being positioned between the light source (11; 21) and the light sensor (12; 22) corresponding to the side of the light source (11; 21) with respect to the tubular portion 2. Such an upstream lens(es) assembly (131; 231) can consist of a single lens having a single focal plane or of a plurality of lenses whose assembly allows defining a global focal plane.

According to one exemplary embodiment, the light source (11; 21) can be positioned at more or less 10 mm from the focal plane of the upstream lens(es) assembly, and preferably in the focal plane of the upstream lens(es) assembly.

Furthermore, each collimation system (13; 23), or at least one of the two, can comprise a downstream lens(es) assembly having a focal plane and being positioned between the corresponding light source (11; 21) and light sensor (12; 22) on the side of the light sensor (12; 22) with respect to the tubular portion 2. Such a downstream lens(es) assembly may consist of a single lens having a single focal plane or of a plurality of lenses whose assembly allows defining a global focal plane.

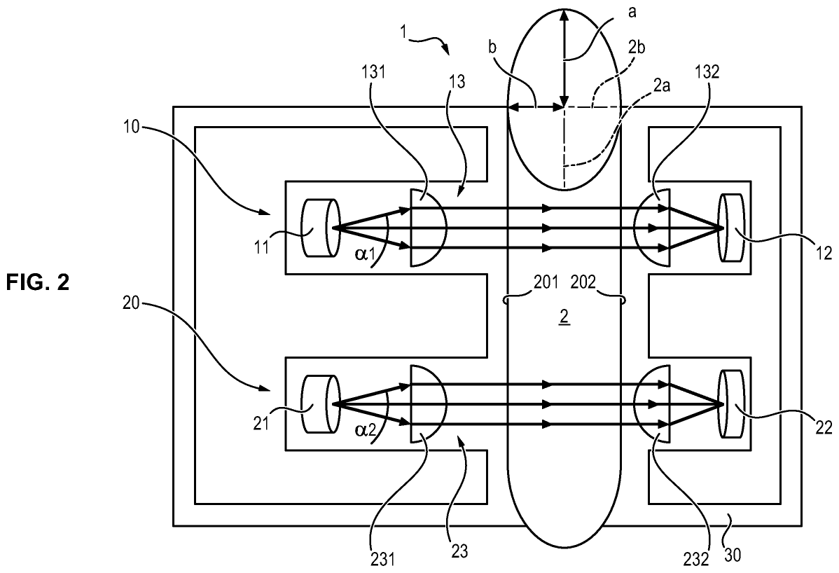

According to one exemplary embodiment illustrated in FIG. 2, the light sensor (12; 22) can be for example positioned at more or less 10 mm from the focal plane of the downstream lens(es) assembly (132; 232), and preferably in the focal plane of the downstream lens(es) assembly (132; 232). Thus, when the light beams leaving the outlet wall 202 of the tubular portion 2 are collimated substantially to infinity, this set of downstream lens(es) allows making these light beams converge on the crresponding light sensor (12; 22).

According to another exemplary embodiment, the downstream lens(es) assembly is positioned so that the light beams leaving the outlet wall 202 of the tubular portion 2 converge at more or less 10 mm from the focal plane of the downstream lens(es) assembly, and preferably in the focal plane of this set of downstream lens(es). Thus, this set of downstream lens(es) allows collimating to infinity the light beams in the direction of the corresponding light sensor (12; 22).

It should be noted that the upstream lens(es) assembly and/or the downstream lens(es) assembly could be mounted in the apparatus 1 so as to be able to be translated along the general optical axis, for example in an automated manner, so as to be able to vary their positioning according to the dimensions and the deformation of the tubular portion 2 in which the fluid whose hematocrit level is to be determined circulates.

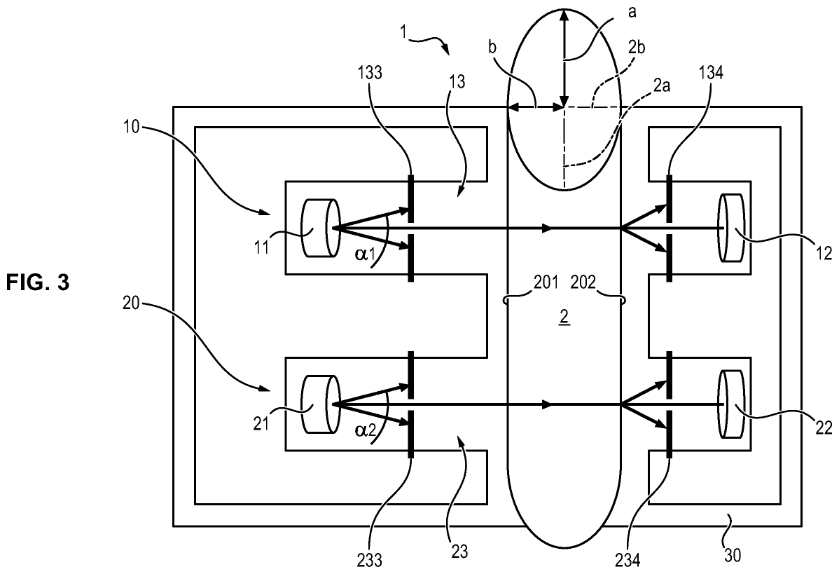

Furthermore, as illustrated in FIG. 3, each collimation system (13; 23), or at least one of the two, can comprise an upstream diaphragm (133; 233) positioned between the corresponding light source (11; 21) and light sensor (12; 22) on the side of the light source (11; 21) with respect to the tubular portion 2. Such an upstream diaphragm (133; 233) is configured and arranged in the apparatus 1 to let pass a central portion of the light beams emitted by the light source (11; 21) in the direction of the light sensor (12; 22) and to stop a peripheral portion of the light beams emitted by the light source (11; 21).

Additionally or as alternatively, as illustrated in FIG. 3, each collimation system (13; 23), or at least one of the two, can comprise a downstream diaphragm (134; 234) positioned between the corresponding light source (11; 21) and light sensor (12; 22) on the side of the light sensor (12; 22) with respect to the tubular portion 2. Such a downstream diaphragm (134; 234) is configured and arranged in the apparatus 1 to let pass a central portion of the light beams transmitted through the tubular portion 2 in the direction of the light sensor (12; 22) and to stop a peripheral portion of the light beams transmitted through the tubular portion 2.

The use of an upstream diaphragm (133; 233) and/or of a downstream diaphragm (134; 234) is particularly advantageous since this allows eliminating the light beams that interfere with the receipt of the light sensors (12; 22) and therefore disturb the measurements of the apparatus 1. Such diaphragms allow for example reducing the noise caused by the light beams reflected, diffracted or scattered by the tubular portion 2. Indeed, the upstream diaphragm (133; 233) allows selecting and centering the light beam emitted from the light source (11; 21) on the tubular portion 2 in order to minimize its scattering and reflection. The downstream diaphragm (134; 234) for its part allows further refining the signal received by the light sensor (12; 22) since it only lets pass the central beams transmitted by the tubular portion 2 while cutting the parasitic beams such as the light beams reflected, diffracted or scattered by the tubular portion 2. Another advantage of the use of diaphragm(s) is that they allow improving the level of receipt by the light sensors (12; 22) without having to increase the power of the light sources (11; 21) which allows increasing the service life of the components of the apparatus 1. It should be noted that the use of diaphragms, in particular of the upstream diaphragm, will be all the more more preferred than the emission cone of the light sources (11; 21) will be narrowed, that is to say the angles (α1; α2) of the emission cones of the light sources (11; 21) will be small, so that a major part of the light beam coming from the light sources (11; 21) is concentrated by cutting only the parasitic peripheral light beams.

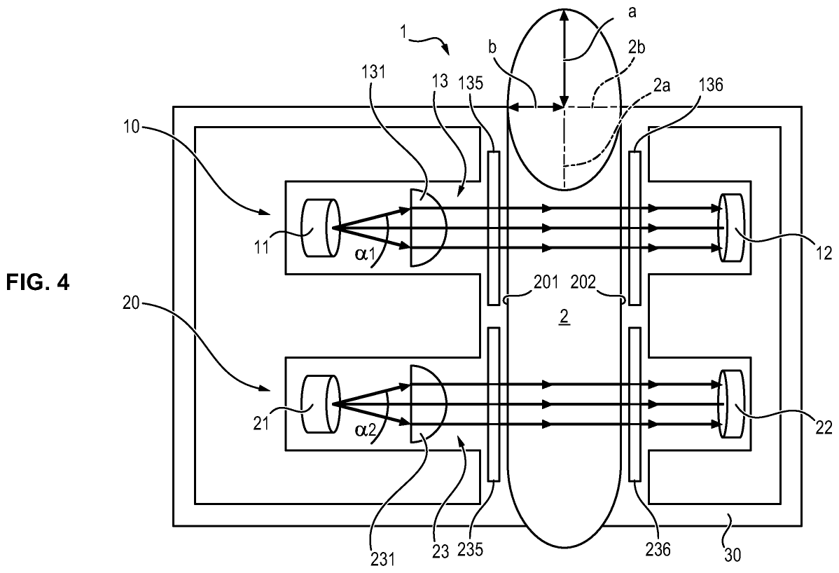

Additionally or alternatively, and as illustrated in FIG. 4, each collimation system (13; 23), or at least one of the two, can comprise an upstream filter (135; 235) positioned between the corresponding light source (11; 21) and light sensor (12; 22) on the side of the light sensor (12; 22) with respect to the tubular portion 2. Such a downstream filter (136; 236) of the collimation system (13; 23) of a transceiver assembly (10; 20) is provided to filter at least the emission wavelength of the light source (11; 21) of the other transceiver assembly (10; 20). Preferably, the downstream filter (136; 236) of the collimation system (13; 23) of a transceiver assembly (10; 20) is provided to block all the light beams not being at the wavelength of interest, that is to say the emission wavelength of the corresponding light source (11; 21).

The use of an upstream filter (135; 235) and/or a downstream filter (136; 236) is particularly advantageous in that it allows limiting the light beam received by a particular light sensor (12; 22) to the sole light beams coming from the corresponding light source (11; 21), while avoiding a disturbance by parasitic light beams coming from the other light source (11; 21), or parasitic beams coming from the ambient light.

As indicated above, it is preferable for the emission cones of the light sources (11; 21) defined by the angles (α1; α2) to be as narrow as possible so that the intensity of the light beam centered on the tubular portion 2 is as high as possible without having to use a too high emission power for the light sources (11; 21) so as not to reduce the service life of the elements forming the apparatus 1.

Thus, the angles (α1; α2) of the emission cones of the light sources (11; 21) is preferably comprised between 1° and 25°, and preferably between 5° and 20°, and more preferably between 10° and 15°. It should be noted that the angle (α1; α2) of the emission cone can depend on the emission wavelength used for the light source (11; 21).

For a light source (11; 21) emitting at a wavelength near 810 nm, the angle (α1; α2) of the emission cone can be for example of the order of 13° (±1°).

For a light source (11; 21) emitting at a wavelength near 1,300 nm, the angle (α1; α2) of the emission cone can be for example of the order of 15° (±1°).

As specified above, the proposed apparatus 1 for determining the hematocrit level is configured to be positioned around an existing tubular portion, for example a flexible tubing or tube used in a hemorrhagic fluid treatment system, so as to allow a determination of the hematocrit level in a non-invasive manner.

To this end, the apparatus 1 comprises a support assembly 30 on which the elements forming the apparatus 1, particularly the transceiver assemblies (10; 20) are mounted. As indicated previously, the support assembly 30 is preferably configured to be positioned around the tubular portion.

Such a support assembly 30 can for example comprise a single support 31 as illustrated in FIGS. 1 to 6, this support 31 having a groove 32 intended to receive the tubular portion 2. The light sources (11; 21) and the light sensors (12; 22) are then arranged on the support 31 on either side of the groove 32.

The support assembly can further comprise a cover 33 provided to at least partially cover the groove 32 of the support 31. Such a cover 33 is provided to prevent the withdrawal of the tubular portion 2 which would be inserted into the groove 32, thus having a lock function.

According to one advantageous embodiment, the cover 33 comprises a compression portion 331 intended to compress the tubular portion 2 positioned in the groove 32. This compression portion 331 is adapted to at least hold in position the tubular portion 2 in the groove 32 of the support 31 of the apparatus 1.

According to the embodiment illustrated in FIG. 6, the support assembly 30 comprises a shell 34 intended to cover the support 30, in particular to protect the elements of the apparatus 1. Such a shell 34 forms the outer casing of the apparatus 1.

Such a cover 33 can be for example mounted in an articulated manner with respect to the support 31. The cover 33 is for example assembled on the shell 34 in an articulated manner and arranged so as to face the groove 32 of the support 31.

Preferably, the cover 33 and/or the shell 34 have outer surfaces for protecting the transceiver assemblies (10; 20) from external disturbances, in particular the ambient light.

The cover 33 and/or the shell 34 also have preferably outer surfaces preventing the reflection of the light rays due to the light sources (11; 21) and not directed towards the light sensors (12; 22), such as for example all scattered, diffracted, reflected rays. Preferably, the outer surfaces of the cover 33 and/or of the shell 34 are provided to absorb these light rays.

The cover 33 and/or the shell 34 can be for example formed in a totally opaque material.

The cover 33 and/or the shell 34 are furthermore preferably provided to guarantee a tightness of the apparatus 1, particularly a fluid tightness in order to protect all the sensitive elements of the apparatus 1, in particular the electronic components.

The fact of having a single support 31 for the apparatus 1 allows a precise mounting and a holding in position of the elements forming the transceiver assemblies (10; 20). Such an embodiment is particularly advantageous since it allows in particular getting as close as possible to the optimum optical conditions for the light beams, particularly with regard to their centering with respect to the tubular portion 2.

Each element forming the transceiver assemblies (10; 20) can be mounted individually on the support 31 in order to form the apparatus 1. The uniqueness of the support 31 allows holding in position the elements with respect to each other but the mounting as such can be difficult. To facilitate the mounting of the elements forming the transceiver assemblies (10; 20) while guaranteeing a precise positioning, it is proposed to use mounting shafts (311; 321; 312; 322) in which the elements forming the transceiver assemblies (10; 20) are pre-mounted, which mounting shafts (311; 321; 312; 322) then being inserted into mounting cavities arranged in the support 31, these mounting cavities having a shape complementary to the mounting shafts (311; 321; 312; 322), allowing for example a forced insertion of the mounting shafts (311; 321; 312; 322) into these mounting cavities.

In each mounting shaft (311; 321; 312; 322) one or more cavities for receiving the elements forming the transceiver assemblies (10; 20) are arranged, each cavity being dimensioned to receive the specific element to be positioned.

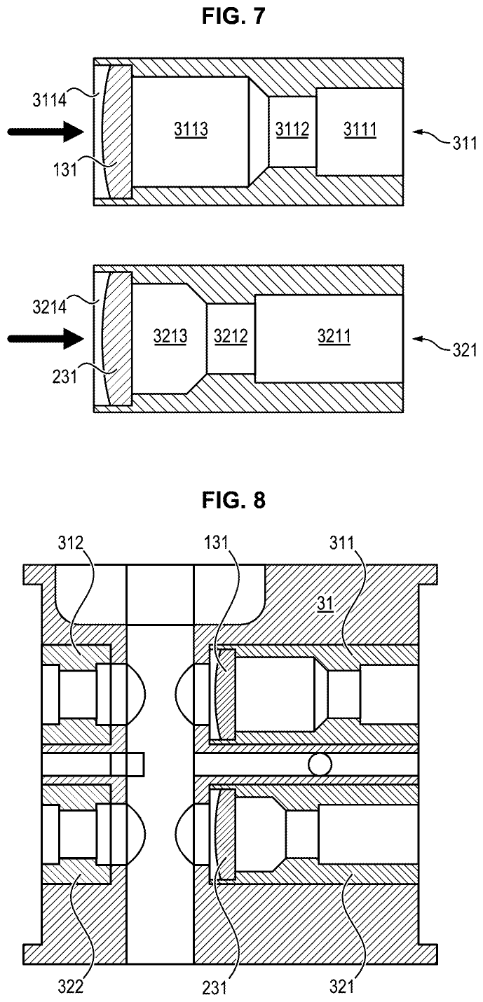

FIG. 7 represents exemplary embodiments of mounting shafts (311; 312) for the elements forming the collimation system (13; 23) of the apparatus 1 according to the example of FIG. 1, and light sources (11; 21). Each mounting shaft (311; 312) has a substantially elongated, preferably cylindrical, shape and comprises several cavities (3111, 3112, 3113, 3114; 3121, 3122, 3123, 3124) connected to each other so as to form a through opening through the mounting shaft (311; 312). Preferably, two adjacent cavities (3111, 3112, 3113, 3114; 3121, 3122, 3123, 3124) have sections of different size and/or shape so that the cooperation of adjacent cavities (3111, 3112, 3113, 3114; 3121, 3122, 3123, 3124) allows forming a space for receiving an element forming the collimation system (13; 23) or the light source (11; 21). The cavities (3111, 3112, 3113, 3114; 3121, 3122, 3123, 3124) are therefore formed in the mounting shaft (311; 312) in order to allow a precise positioning of the elements forming the transceiver assemblies (10; 20).

According to the embodiment illustrated in FIG. 7, the collimation system (13; 23) comprises a lens (131; 231) which can be inserted into the end cavity (3114; 3214) and abut against the shoulder formed by the adjacent cavity (3113; 3213).

The cavity (3113; 3213) on which the lens (131; 231) abuts has a length corresponding to the desired distance between the lens (131; 231) and the corresponding light source (11; 21). Preferably, this length is chosen so that the light source (11; 21) is in the focal plane of the lens (131; 231). This cavity (3113; 3213) has therefore a remote hold function.

The light source (11; 21) is for its part intended to be inserted from the other end cavity (3111; 3211) up to the adjacent cavity (3112; 3212), this cavity (3112; 3212) also being adjacent to the remote holding cavity (3113; 3213). The light source (11; 21) can for example comprise a protuberance abutting against a shoulder formed between the cavity (3112; 3212) for receiving the light source (11; 21) and the end cavity (3111; 3211).

Once the lens (131; 231) is pre-mounted in the mounting shaft (311; 312), it is possible to insert this mounting shaft (311; 312) into the mounting cavity provided for this purpose in the support 30. FIG. 8 illustrates this insertion of the mounting shafts (311; 321; 312; 322) into the mounting cavities provided for this purpose in the support 31.

According to the example illustrated in FIG. 8, the light sources (11; 21) and the light sensors (12; 22) are mounted in the cavities arranged in the mounting shafts (311; 321; 312; 322) once these mounting shafts (311; 321; 312; 322) have been inserted into the support 30. It can however be provided to pre-mount the light sources (11; 21) and the light sensors (12; 22) in the cavities arranged in the mounting shafts (311; 321; 312; 322) before these mounting shafts (311; 321; 312; 322) are inserted into the support 31.

According to one alternative arrangement (not represented), the support assembly 30 comprises two supports intended to be assembled with each other by surrounding the tubular portion 2.

An upstream support can thus be provided, on which are arranged the light sources (11; 21) and all elements of the transceiver assemblies (10; 20) provided to be on the side of the corresponding light source (11; 21) with respect to the tubular portion 2.

A downstream support distinct from the upstream support is also provided, on which are arranged the light sensors (12; 22) and all elements of the transceiver assemblies (10; 20) provided to be on the side of the corresponding light sensor (12; 22). with respect to the tubular portion 2.

Preferably, the downstream and upstream supports have complementary shapes provided to be coupled so as to enclose the tubular portion 2.

One of the important characteristics of the apparatus 1 proposed is that the flow of the fluid circulating in the tubular portion 2 is not or little modified so as not to have a negative impact on this fluid. For example, for a hemorrhagic fluid such as blood, an excessive modification of the flow due for example to a substantial constriction of the tubular portion 2 at the detection area of the apparatus 1 could create hemolysis, which is to be avoided for an effective treatment of the hemorrhagic fluid. Particularly, it is not desired for the tubular portion 2 at the detection area to be flattened such that the inlet wall 201 and the outlet wall 202 are substantially parallel to each other since it would create too much hemolysis for the treatment of the hemorrhagic fluid. Thus the transceiver assemblies (10; 20) are preferably arranged in the apparatus 1 for a measurement in transmission through the bent walls of the tubular portion 2, that is to say curved walls.

The simplest way to avoid a risk of hemolysis is not to deform the tubular portion 2. The high curvatures of the circular section of the tubular portion 2 can however disturb the transmission of the light beams from the light sources (11; 21) to the light sensors (12; 22). Thus, it can be envisaged to slightly deform the tubular portion 2, in a monitored manner (for example with a compression rate of the order of 2%), so as not to or little disturb the flow of the fluid in the tubular portion 2 while reducing the curvatures of the tubular portion 2 to reduce their effect on the orientation of the light beams passing through the tubular portion 2 and thus increase the measurement accuracy of the apparatus 1. It should be noted here that the specific arrangement of the apparatus 1 and in particular the use of collimation systems (13; 23) in the transceiver assemblies (10; 20) allows having reliable detection including when the tubular portion 2 has a certain curvature at the detection area. This is why it is not necessary to have a flattening of the tubular portion 2 at this detection area.

Preferably, the apparatus and the method proposed are provided for a determination of the hematocrit level and/or the hemoglobin level of the fluid circulating without deformation of the tubular portion.

Regardless of the support assembly 30 used for the apparatus 1, a system for deforming the tubular portion 2 positioned facing the transceiver assemblies (10; 20) can however be provided.

Preferably, the tubular portion at which the measurement in transmission is performed retains a certain curvature, and is therefore not flattened.

According to one preferred embodiment, the deformation system is provided to deform the circular section of the tubular portion into an ellipsoidal section. To this end, the deformation system can for example use the cooperation of the cover 33, and more specifically of the compression portion 331, with the shape of the groove 32. The groove 32 can indeed have a section of substantially ellipsoidal shape and the compression portion 331 is provided to compress the tubular portion 2 so that it deforms and substantially matches the shape of the groove 32.

The ellipsoidal section of the deformed tubular portion 2 is defined by a major axis 2a and a minor axis 2b perpendicular to the major axis. Preferably, the deformation is such that the light sources (11; 21) and all elements of the transceiver assemblies (10; 20) provided to be on the side of the corresponding light source (11; 21) with respect to the tubular portion 2 are positioned on one side of the major axis 2a, and the light sensors (12; 22) and all elements of the transceiver assemblies (10; 20) provided to be on the side of the corresponding light sensor (12; 22) with respect to the tubular portion 2 are positioned on the other side of the major axis 2a.

The ellipsoidal section of the deformed tubular portion 2 is further defined by a large radius (Ra) along the major axis 2a and by a small radius (Rb) along the minor axis 2b, the ellipsoidal section having, in a deformed state of the tubular portion, a small radius (Rb) having a length comprised between 30% and 70%, and preferably of the order of 50%, of the radius of the circular section of the tubular portion 2 in the undeformed state.

The transceiver assemblies (10; 20) are provided to be coupled to a central processing unit making it possible to drive them, both in emission and in receipt, but also making it possible to process the information from the transceiver assemblies (10; 20).

The apparatus 1 can for example comprise a monitoring system connected to the central processing unit and configured to control the light sources (11; 21) of the transceiver assemblies (10; 20).

The apparatus 1 can further comprise a processing system connected to the central processing unit and configured to recover and process the signals coming from the light sensors (12; 22) of the transceiver assemblies (10; 20), in order in particular to determine the hematocrit level of the circulating fluid.

The light sources (11; 21) and the light sensors (12; 22) of the transceiver assemblies (10; 20) are thus preferably connected to an electronic circuit 40 allowing the monitoring system to control the light sources (11; 21) on the one hand, and the processing system to recover the signals received by the light sensors (12; 22) on the other hand.

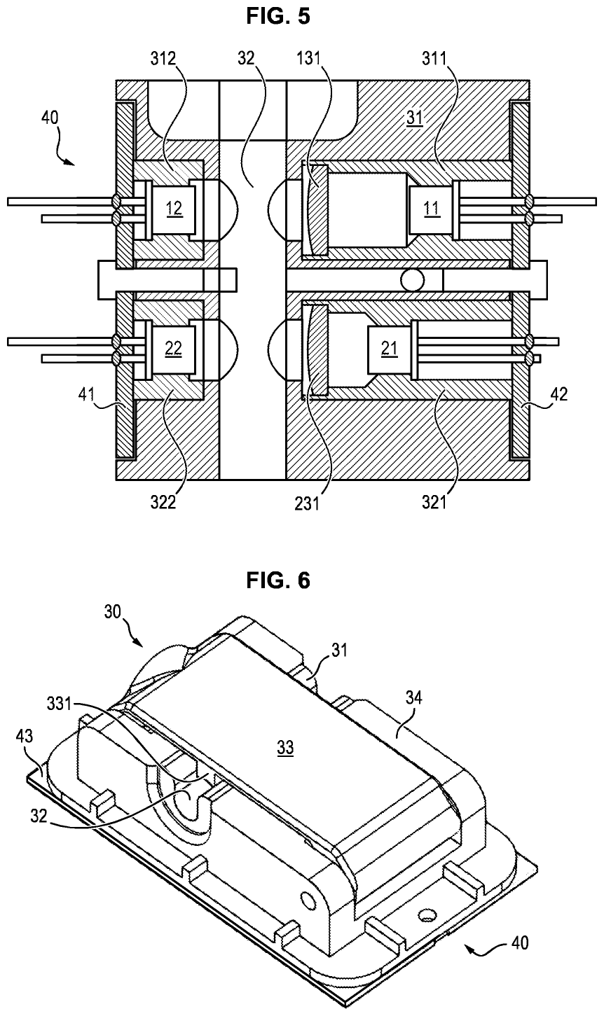

According to the example illustrated in FIGS. 5, 7 and 8, the electronic circuit 40 comprises a first electronic card 41 intended to be connected to the light sources (11; 21) and a second electronic card 42 intended to be connected to the light sensors (12; 22). Each of these first and second electronic cards (41; 42) is preferably coupled to the light sources (11; 21) and to the light sensors (12; 22) respectively once they have been mounted in the support 31.

According to this embodiment, the electronic circuit 40 further comprises a third electronic card 43 connecting the first and second electronic cards (41; 42). This third electronic card 43 can furthermore form a wall of the apparatus 1 forming with the shell 34 the outer casing of the apparatus 1.

It could be envisaged to monitor the light sources (11; 21) so that they emit alternately with each other, in particular so as to reduce the possible interference between the two transceiver assemblies (10; 20). The specific configuration of the proposed apparatus 1 however allows not requiring this emission alternation since other solutions are provided to avoid this interference between the transceiver assemblies (10; 20).

Thus, the monitoring system is preferably configured so that the light sources (11; 21) emit at the same time, that is to say concomitantly. This allows for example having continuous measurements, which allows getting as close as possible to a real-time and continuous detection. This also allows increasing the reliability of the detection since it is possible to correlate the detection of the two light sensors (12; 22) at the same time t, and not one at a time t and the other at a time t+n. The correlation is furthermore simplified. The monitoring system of the apparatus 1 can thus comprise means for synchronizing the light sources (11; 21), the monitoring system therefore being configured to synchronize the emission of the light sources (11; 21).

As will be seen in detail below, it can be advantageous to modify the emission power of the light sources (11; 21) during the process of determining the hematocrit level of the fluid circulating in the tubular portion 2. To this end, the monitoring system can therefore comprise means for modifying the power emitted by the light sources (11; 21), the monitoring system therefore being configured to modify the power emitted by the light sources (11; 21). This modification of the emission power of the light sources (11; 21) can for example depend on the value of the hematocrit level detected for the fluid circulating in the tubular portion 2.

Operation of the Apparatus for the Determination of the Hematocrit Level of a Circulating Fluid

The light signals received by the light sensors (12; 22) of the transceiver assemblies (10; 20) are intended to be processed by the processing system to determine the hematocrit level of the fluid circulating in the tubular portion around which the apparatus 1 has been positioned.

The apparatus 1 for the determination of the hematocrit level of a circulating fluid therefore operates according to the following steps:
emitting light beams in the direction of the tubular portion with the light sources which are configured to emit light beams according to an emission wavelength chosen to correspond to an isosbestic point of hemoglobin;
receiving the light signals transmitted through the tubular portion and the fluid circulating with the light sensors being associated with the light sources respectively;
determining the hematocrit level measured for the fluid by a processing of the light signals received by the sensors, in particular by a calculation performed by the processing system.

There are different correlative calculation methods to determine the hematocrit level as a function of the light signals coming from light sources (11; 21) emitting according to the emission wavelength chosen to correspond to an isosbestic point of hemoglobin.

It is for example possible to use the formula proposed in the article entitled “Noninvasive and Continuous Hematocrit Measurement by Optical Method without Calibration” published by SHOTA EKUNI and YOSHIYUKI SANKAI in “Electronics and Communications in Japan, Vol. 99, No. 9, 2016”.

According to this method, the hematocrit level is calculated as follows: it is known that the concentration of a light-absorbing substance and the intensity of the transmitted light passing through the substance have a logarithmic relationship. The present method applies for a scattering measurement by integrating the two transceiver assemblies (10; 20) operating at the wavelengths λ1 and λ2 and allows determining the value of the variable D_{pw} according to the following formula:

$${D}_{pw} = {{log}_{10} ( \frac{I}{I - Δ I} )}_{λ 1} - {{log}_{10} ( \frac{I}{I - Δ I} )}_{λ 2}$$

Where ΔI is the difference in the light intensity transmission between the two receivers;
Where [log₁₀ (I/(I−ΔI)_{λ1}] and [log₁₀ (I/(I−ΔI)_{λ2}] are the differences in the intensity of the scattered light at the wavelengths λ1 and λ2.

The value D_{pw} obtained is a function of the hematocrit level linearly.

The adaptation of this formula to the apparatus for determining the hematocrit level operating in transmission allows determining the value D_{pw} according for example to the following formula:

$${D}_{pw} = {{log}_{10} ( \frac{I}{{I}_{0}} )}_{λ 1} - {{log}_{10} ( \frac{I}{{I}_{0}} )}_{λ 2}$$

Where I₀ is the blank value recorded during the calibration for the transceiver assemblies (10; 20) of the wavelengths λ1 and λ2;
Where [Log₁₀ I] is the logarithm of the intensity of the transmitted light for the wavelengths λ1 and λ2.

The obtained value D_{pw} is also a function of the hematocrit level linearly.

It has been observed that the determination of the hematocrit level by these calculation methods can vary and at times be unreliable depending on the hematocrit level of the circulating fluid. Particularly, cases were detected where the calculations could be distorted for the low hematocrit levels (typically below 20%) and/or for the high hematocrit levels (typically above 50%).

However, it may be necessary to have an apparatus 1 for determining the hematocrit level that operates reliably for a wide range of hematocrit levels, which is particularly advantageous when the apparatus 1 is used, for example, in a hemorrhagic fluid treatment assembly where the hemorrhagic fluid to be treated generally has a low hematocrit level (typically lower than 20% or even lower than 10%) before starting the treatment while the target hematocrit level to be achieved for the treated hemorrhagic fluid is high, for example by at least 35%, even at least 45%, and sometimes at least 50%.

To make the determination of the hematocrit level more reliable whatever the value of this hematocrit level, it is proposed to be able to modify the emission power of at least one of the light sources (11; 21) of the transceiver assemblies (10; 20) during the measurement of the hematocrit level as a function of the hematocrit level calculated for the fluid.

Preferably, the monitoring system is provided to modify the emission power of all the light sources (11; 21) of the transceiver assemblies (10; 20) during the measurement of the hematocrit level as a function of the hematocrit level calculated for the fluid.

Advantageously, the emission power of the light sources (11; 21) is monitored independently for each of the light sources (11; 21). This independent monitoring is particularly advantageous when the light sources (11; 21) are different, in particular when the emission wavelengths are different.

It should first be noted that it is advantageous not to use the light sources (11; 21) at 100% of their capacity. Indeed, it is preferable to use the light sources (11; 21) at an emission power lower than the maximum power of the light sources (11; 21) to increase the longevity of the apparatus 1 on the one hand, but also to avoid a possible degradation of the elements of the apparatus 1, or a heating of the circulating fluid to be analyzed for example.

To increase the sensitivity of the apparatus 1 whatever the value of the hematocrit level of the circulating fluid, and without having to modify the parameters of the light sensor (12; 22), it is furthermore advantageous to vary the emission power of the light sources (11; 21) as a function of the hematocrit level. Particularly, the higher the hematocrit level, the greater the risk of the light signal coming from the light sources (11; 21) being absorbed by the circulating fluid, which can be compensated by an increase in the emission intensity of the light sources (11; 21) for similar levels of receiving intensities at the light sensors (12; 22).

The emission power of the light sources (11; 21) is therefore driven as a function of the detection level of the light sensors (12; 22) correlated with the measured hematocrit level.

Particularly, the emission power of the light sources (11; 21) can be driven according to the non-linearity threshold below which the value measured by the light sensors (12; 22) does not allow calculating the hematocrit level with sufficient reliability.

Particularly, it is advantageous to drive the emission power of the light sources (11; 21) so that the signal received has a power greater than the non-linearity threshold but as close as possible to this non-linearity threshold, while being of a sufficient level as a function of the measured hematocrit level.

Alternatively or additionally, the emission power of the light sources (11; 21) can be driven according to the saturation threshold of the light sensor (12; 22) beyond which the light signal received at the light sensor (12; 22) is not measurable.

In practice, even if it is possible to use each light source (11; 21) at 100% of their maximum emission power level, it is advantageous to use an emission power for the light sources (11; 21) comprised between 10% and 60% of the maximum emission power of said light sources.

The apparatus 1 proposed is provided to allow a determination of a wide range of hematocrit level, particularly both for hematocrit levels as low as 5% or even lower than 5%, and for high hematocrit levels of the order of 50% even of the order of 60% or higher.

During use of the apparatus 1 for determining the hematocrit level of a circulating fluid, it is advantageous to gradually increase the emission power of at least one of the light sources (11; 21), and preferably all the light sources (11; 21), particularly as the hematocrit level of the fluid increases. Preferably, the emission power of at least one of the light sources (11; 21) is increased from a threshold value of the hematocrit level measured for the fluid.

When the measured hematocrit level is lower than 30%, the emission power of at least one of the light sources (11; 21), and preferably of all the light sources (11; 21), can be for example set to a value comprised between 10% and 30%, preferably substantially equal to 20%, of the maximum emission power of the corresponding light source.

When the measured hematocrit level is higher than or equal to 30%, the emission power of at least one of the light sources (11; 21), and preferably of all the light sources (11; 21), can be for example set to a value comprised between 30% and 100%, preferably substantially equal to 50%, of the maximum emission power of the corresponding light source.

According to one particular embodiment, the emission power of at least one of the light sources (11; 21), and preferably of all the light sources (11; 21), is adjusted so that:
the emission power of said light source is at a first power level for values of the hematocrit level measured for the fluid lower than a first threshold value;
the emission power of said light source is at a second power level for values of the hematocrit level measured for the fluid higher than or equal to the first threshold value but lower than a second threshold value higher than the first threshold value; and
the emission power of said light source is at a third power level for values of the hematocrit level measured for the fluid higher than or equal to the second threshold value.

According to this embodiment, a specific example of monitoring of the light source(s) (11; 21) is as follows:
the emission power of the light source is set to a value comprised between 5% and 15%, preferably equal to 10%, of the maximum emission power of said light source when the measured hematocrit level is lower than 20%;
the emission power of the light source is set to a value comprised between 15% and 30%, preferably equal to 20%, of the maximum emission power of said light source when the measured hematocrit level is comprised between 20% and 30%; and
the emission power of the light source is set to a value comprised between 30% and 100%, preferably equal to 55%, of the maximum emission power of said light source when the measured hematocrit level is higher than or equal to 30%.

The emission power of the light sources can also be modified during the measurement of the hematocrit level depending on the presence or absence of fluid in the tubular portion 2 and/or on the nature of said fluid. Particularly, if the tubular portion is devoid of fluid, it is preferable that the light source(s) (11; 21) are maintained at a minimum, or even zero, emission level.

### BIBLIOGRAPHICAL REFERENCES

“Noninvasive and Continuous Hematocrit Measurement by Optical Method without Calibration” from SHOTA EKUNI and YOSHIYUKI SANKAI (Electronics and Communications in Japan, Vol. 99, No. 9, 2016)

1. A method for determining the hematocrit level and/or the hemoglobin level of a fluid circulating in a tubular portion (2), comprising:
the emission of light beams in the direction of the tubular portion (2) with at least two light sources (11; 21), each of the two light sources (11; 21) being configured to emit light beams according to an emission wavelength chosen to correspond to an isosbestic point of hemoglobin;
the receipt of light signals transmitted through the tubular portion (2) with at least two light sensors (12; 22), each light sensor (12; 22) being associated with one of the two light sources (11; 21);
the calculation of the hematocrit level and/or the hemoglobin level of the fluid by a processing of the light signals received by the light sensors (12; 22);
characterized in that the emission power of at least one of the light sources (11; 21) is modified during the determination of the hematocrit level and/or the hemoglobin level as a function of the hematocrit level and/or of the hemoglobin level respectively calculated for the fluid.

2. The method of claim 1, wherein the emission power used for the light sources (11; 21) is at most equal to 100% of the maximum emission power of said light sources (11; 21), and preferably comprised between 10% and 60% of the maximum emission power of said light sources (11; 21).

3. The method of any one of claims 1 and 2, wherein the emission power of the light sources (11; 21) is monitored independently for each of the light sources (11; 21).

4. The method of any one of claims 1 to 3, wherein the emission power of at least one of the light sources (11; 21) is increased from a threshold value of the hematocrit level and/or the hemoglobin level calculated for the fluid.

5. The method of claim 4, wherein:
the emission power of the light source (11; 21) is set to a value comprised between 10% and 30%, preferably equal to 20%, of the maximum emission power of said light source (11; 21) when the calculated hematocrit level is lower than 30%; and
the emission power of the light source (11; 21) is set to a value comprised between 30% and 100%, preferably equal to 55%, of the maximum emission power of said light source (11; 21) when the calculated hematocrit level is higher than or equal to 30%.

6. The method of any one of claims 1 to 5, wherein the emission power of at least one of the light sources (11; 21) is adjusted so that:
the emission power of said light source (11; 21) is at a first power level for values of the hematocrit level calculated for the fluid lower than a first threshold value;
the emission power of said light source (11; 21) is at a second power level for values of the hematocrit level calculated for the fluid higher than or equal to the first threshold value but lower than a second threshold value higher than the first threshold value; and
the emission power of said light source (11; 21) is at a third power level for values of the hematocrit level calculated for the fluid higher than or equal to the second threshold value.

7. The method of claim 6, wherein the emission power of at least one of the light sources (11; 21) is adjusted so that:
the emission power of the light source (11; 21) is set to a value comprised between 5% and 15%, preferably equal to 10%, of the maximum emission power of said light source (11; 21) when the calculated hematocrit level is lower than 20%;
the emission power of the light source (11; 21) is set to a value comprised between 15% and 30%, preferably equal to 20%, of the maximum emission power of said light source (11; 21) when the calculated hematocrit level is comprised between 20% and 30%; and
the emission power of the light source (11; 21) is set to a value comprised between 30% and 100%, preferably equal to 55%, of the maximum emission power of said light source (11; 21) when the calculated hematocrit level is higher than or equal to 30%.

8. The method of any one of claims 1 to 7, wherein the emission power of the light sources (11; 21) is modified during the determination of the hematocrit level and/or the hemoglobin level depending on the presence or absence of fluid in the tubular portion (2) and/or on the nature of said fluid.

9. The method of any one of claims 1 to 8, wherein the light sources (11; 21) are monitored to emit light beams concomitantly.

10. An apparatus for determining the hematocrit level and/or the hemoglobin level of a fluid circulating in a tubular portion (2), comprising:
two transceiver assemblies (10; 20), each transceiver assembly (10; 20) comprising a light source (11; 21) and a light sensor (12; 22) provided to be arranged on either side of the tubular portion (2) at a fluid circulation area for a measurement in transmission;
the light source (11; 21) of each of the two transceiver assemblies (10; 20) being configured to emit light beams according to an emission wavelength chosen to correspond to an isosbestic point of hemoglobin;
a processing system programmed to determine the hematocrit level and/or the hemoglobin level of the fluid as a function of the light signals received by the light sensors (12; 22) of the transceiver assemblies (10; 20); and
a monitoring system comprising means for modifying the power emitted by the light sources (11; 21), the monitoring system being programmed to modify the emission power of the light sources (11; 21) as a function of the hematocrit level and/or the hemoglobin level determined for the fluid.

11. The apparatus of claim 10, comprising a support assembly (30) on which the two transceiver assemblies (10; 20) are mounted, the support assembly (30) being configured to be positioned around the tubular portion (2).

12. The apparatus of any one of claims 10 and 11, wherein the respective light sources (11; 21) of the two transceiver assemblies (10; 20) are configured to emit light beams at two different emission wavelengths.

13. The apparatus of any one of claims 10 to 12, wherein at least one of the light sources (11; 21) of the transceiver assemblies (10; 20) is configured to emit light beams according to an emission wavelength chosen for an absorption of the light beams substantially identical in water or in plasma.

14. The apparatus of any one of claims 10 to 13, wherein at least one, and preferably each, collimation system (13; 23) comprises an upstream lens(es) assembly (131; 231) having a focal plane and being positioned between the corresponding light source (11; 21) and light sensor (12; 22) on the side of the light source (11; 21) with respect to the tubular portion (2), the light source (11; 21) being positioned at more or less 10 mm from the focal plane of the upstream lens(es) assembly (131; 231), and preferably in the focal plane of the upstream lens(es) assembly (131; 231).

15. The apparatus of any one of claims 10 to 14, wherein at least one, and preferably each, collimation system (13; 23) comprises a downstream lens(es) assembly (132; 232) having a focal plane and being positioned between the corresponding light source (11; 21) and light sensor (12; 22) on the side of the light sensor (12; 22) with respect to the tubular portion (2), the light sensor (12; 22) being positioned at more or less 10 mm from the focal plane of the set of downstream lens(s) (132; 232), and preferably in the focal plane of the set of downstream lens(s) (132; 232).

16. The apparatus of any one of claims 10 to 15, wherein at least one, and preferably each, collimation system (13; 23) comprises a downstream lens(es) assembly (132; 232) having a focal plane and being positioned between the corresponding light source (11; 21) and light sensor (12; 22) on the side of the light sensor (12; 22) with respect to the tubular portion (2), the downstream lens(es) assembly) (132; 232) is positioned so that the light beams leaving the outlet wall (202) of the tubular portion (2) converge at more or less 10 mm from the focal plane of the downstream lens(es) assembly, and preferably in the focal plane of the downstream lens(es) assembly (132; 232).

17. The apparatus of any one of claims 10 to 16, wherein at least one, and preferably each, collimation system (13; 23) comprises an upstream diaphragm (133; 233) positioned between the corresponding light source (11; 21) and light sensor (12; 22) on the side of the light source (11; 21) with respect to the tubular portion (2), the upstream diaphragm (133; 233) being provided to let pass a central portion of the light beams emitted by the light source (11; 21) in the direction of the light sensor (12; 22) and to stop a peripheral portion of the light beams emitted by the light source (11; 21).

18. The apparatus of any one of claims 10 to 17, wherein at least one, and preferably each, collimation system (13; 23) comprises a downstream diaphragm (134; 234) positioned between the corresponding light source (11; 21) and light sensor (12; 22) on the side of the light sensor (12; 22) with respect to the tubular portion (2), the downstream diaphragm (134; 234) being provided to let pass a central portion of the light beams transmitted through the tubular portion (2) in the direction of the light sensor (12; 22) and to stop a peripheral portion of the light beams transmitted through the tubular portion (2).

19. The apparatus of any one of claims 10 to 18, wherein at least one, and preferably each, collimation system (13; 23) comprises an upstream filter (135; 235) positioned between the corresponding light source (11; 21) and light sensor (12; 22) on the side of the light source (11; 21) with respect to the tubular portion (2), and/or a downstream filter (136; 236) positioned between the corresponding light source (11; 21) and light sensor (12; 22) on the side of the light sensor (12; 22) with respect to the tubular portion (2), the upstream (135; 235) and downstream (136; 236) filters of the collimation system (13; 23) of a transceiver assembly (10; 20) being provided to filter at least the emission wavelength of the light source (11; 21) of the other transceiver assembly (10; 20).

20. The apparatus of any one of claims 10 to 19, wherein:
the light source (11; 21) of a first of the two transceiver assemblies (10; 20) is configured to emit light beams at a wavelength comprised between 780 nm and 840 nm, preferably comprised between 800 nm and 820 nm, and more preferably equal to 810 nm; and
the light source (11; 21) of a second of the two transceiver assemblies (10; 20) is configured to emit light beams at a wavelength comprised between 1,270 nm and 1,330 nm, preferably between 1,290 nm and 1,310 nm, and more preferably equal to 1,300 nm.

21. The apparatus of any one of claims 10 to 20, wherein the light sources (11; 21) of the transceiver assemblies (10; 20) are positioned on the same side with respect to the tubular portion (2).

22. The apparatus of any one of claims 10 to 21, further comprising a system for monitoring the transceiver assemblies (10; 20), the monitoring system comprising means for synchronizing the light sources (11; 21) and/or means for modifying the power emitted by the light sources (11; 21).

23. The apparatus of any one of claims 10 to 22, wherein the transceiver assemblies (10; 20) are assembled on a single support (31) having a groove (32) intended to receive the tubular portion (2).

24. The apparatus of claim 23, further comprising a cover (33) provided to at least partially cover the groove (32), said cover (33) comprising a compression portion intended to hold in position the tubular portion (2) positioned in the groove (32).

25. The apparatus of any one of claims 10 to 22, wherein the light sources (11; 21) and all elements of the transceiver assemblies (10; 20) provided to be on the side of the corresponding light source (11; 21) with respect to the tubular portion (2) are assembled on an upstream support, and the light sensors (12; 22) and all elements of the transceiver assemblies (10; 20) provided to be on the side of the corresponding light sensor (12; 22) with respect to the tubular portion (2) are assembled on a downstream support distinct from the upstream support, the downstream and upstream supports having complementary shapes provided to be coupled so as to enclose the tubular portion (2).

26. The apparatus of any one of claims 10 to 25, provided for a determination of the hematocrit level and/or the hemoglobin level without deformation of the tubular portion (2).

27. The apparatus of any one of claims 10 to 25, comprising a system for deforming the tubular portion (2) facing the transceiver assemblies (10; 20), the deformation system being provided to deform a circular section of the tubular portion (2) into an ellipsoidal section.

28. The apparatus of claim 27, wherein the light sources (11; 21) and all elements of the transceiver assemblies (10; 20) provided to be on the side of the corresponding light source (11; 21) with respect to the tubular portion (2) are positioned on one side of a major axis defining the ellipsoidal section, and the light sensors (12; 22) and all elements of the transceiver assemblies (10; 20) provided to be on the side of the corresponding light sensor (12; 22) with respect to the tubular portion (2) are positioned on the other side of the major axis defining the ellipsoidal section.

29. The apparatus of claim 28, wherein the ellipsoidal section is defined by a major radius (Ra) along the major axis and by a minor radius (Rb) along a minor axis perpendicular to the major axis, the ellipsoidal section having, in a deformed state of the tubular portion (2), a small radius (Rb) having a length comprised between 30% and 70%, and preferably of the order of 50%, of the radius of the circular section of the tubular portion (2) in an undeformed state.
